(19) United States
(12) Patent Application Publication
(10) Pub. No.: US 2009/0226492 A1
(43) Pub. Date: Sep. 10, 2009

(54) USE OF AN ACTIVATED ZEOLITE AS A PHARMACEUTICAL AGENT FOR REDUCING TOXIC SUBSTANCES

(76) Inventors: Hubert Danz, Eilensedt (DE); Thomas Gorner, Schlanstedt (DE); Steffen Hoffmann, Schlanstedt (DE); Oliver Woge, Diemelstadt (DE)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/092,460

(22) PCT Filed: Nov. 4, 2006
(86) PCT No.: PCT/DE2006/001951

§ 371 Date: Jan. 29, 2009

(30) Foreign Application Priority Data
Nov. 4, 2005 (DE) 10 2005 053 090.7

(51) Int. Cl. A61K 8/02; A61K 9/14; A61K 33/06

(52) U.S. Cl. 424/401; 424/489; 424/684

(57) ABSTRACT

The invention relates to the use of an activated and modified zeolite as a pharmaceutical agent for diseases of the metabolic organs, particularly the liver, kidneys and pancreas by adsorbing heavy metals such as lead and mercury and/or ammonia in the digestive tract of humans and animals and for their evacuation from the organism. The particles of the zeolite have a particle size ranging from 2 to 9 μm, zeta potentials ranging from 20 to 26 mV and a specific surface area ranging from 30 to 38 m²/g. Additional applications include use in cases of food intolerance and food allergies, for treating open or closed wounds in a powder or cream form, for skin diseases and viral infections, for skin care and skin regeneration, as cosmetic agents for skin care and skin regeneration, and for taking up and evacuating mercury and amalgam in the tooth and jawbone region of humans.

The invention relates to the use of an activated zeolite as a pharmaceutical agent for reducing undesired toxic substances in the human or animal organism.

Harmful environmental influences and foods and delicacies burden the body with diverse substances that cannot be degraded by the body, build up in the organism over time and can lead directly or indirectly to ill health and overstress of the metabolic organs.

Similarly, heavy-metal burdens come about through dental treatments, for example upon the removal of amalgam fillings or the like.

The adsorption capacity of zeolites generally and for heavy-metal ions in particular has long been known to science.

According to EP 1 316 530 B1, the use of zeolites 0.5 μm in size for use as a pharmaceutical agent is commonly known. Applications are described for the treatment of metabolic disorders as well as cardiovascular disorders as well as rheumatic diseases and multiple sclerosis as well as dermatological diseases. According to the publication cited, the pharmaceutical actions result from the fineness of the zeolite particles, whose size renders them resorbable in the human digestive tract.

Also associated therewith is a disadvantage of the cited prior art, because zeolites are partly taken up by the organism and reside therein. A further disadvantage is that the claimed detoxification of the human organism often involves coadsorption and deactivation of important substances such as for example zinc, and thus the body may exhibit deficiencies of substances upon application or prolonged consumption.

It is an object of the invention to furnish a pharmaceutical agent for effective detoxification of the human body in respect of heavy metals and ammonium.

The object of the invention is achieved through the use of a zeolite reduced in size and activated in special fashion (MAC), which exhibits certain surface properties. The substance according to the invention is also referred to as modified and activated zeolite.

The particles of the zeolite exhibit a size of 6 to 9 μm. The zeta potentials of the zeolite vary over a range of 24 to 26 mV. Its specific surface area is 35 to 36 m²/g.

The concept of the invention consists in that the modified and activated zeolite exhibits a particle size and surface properties that permit optimal adsorption of the undesired constituents and in that the zeolites adsorptively burdened with the undesired substances can subsequently be discharged from the organism. The latter is advantageously achieved in that the particles exhibit a size according to the invention and cannot be resorbed by the organism.

According to the invention, the adsorbability of heavy metals and the fixing of ammonium are thus applied to relieving a burden on the metabolism. The zeolite can be used according to the invention both in the digestive tract of the human or animal organism and also upon external application.

A naturally occurring zeolite, clinoptilolite, is employed according to the invention. Clinoptilolites are hydrated aluminosilicates (zeolites) having a net formula (Ca,K₂,Na₂,Mg)₄Al₈Si₄₀O₉₆.24H₂O. The clinoptilolite according to the invention is a calcium aluminum silicate.

Natural occurrences comprise up to 84% pure clinoptilolite. The balance of the mineralogical composition is made up by:

| | |
|---|---|
| Cristobalite | 8% |
| Feldspar | 3-4% |
| Illite | 4% |
| Quartz | traces |
| Carbonate minerals | |

Chemically, the mineral is composed of the following oxides:

| | |
|---|---|
| SiO₂ | 65-71.3% |
| Al₂O₃ | 11.5-13.1% |
| CaO | 2.7-5.2% |
| K₂O | 2.2-3.4% |
| Fe₂O₃ | 0.7-1.9% |
| MgO | 0.6-1.2% |
| Na₂O | 0.2-1.3% |
| TiO₂ | 0.1-0.3% |

The following physical data were determined:

| Softening point | 1260° | C. |
|---|---|---|
| Melting point | 1340° | C. |
| Density | 2.2-2.5 | g/cm³ |
| Compressive strength | 33 | MPa |
| Porosity | 32-40% | |
| Pore diameter | 0.4 | nm |

| | |
|---|---|
| Mohs hardness | 2.5-3.5 |

The clinoptilolite is gray-green in color and odorless.

The skeleton of the clinoptilolite crystal lattice comprises SiO₂ tetrahedra and Al₂O₃. The clinoptilolite has a silicon:aluminum ratio of around 4:1. The crystalline structures formed via oxygen bridges allow pores and channels to form.

Cation-exchange capacity results from the replacement of tetravalent silicon atoms by trivalent aluminum atoms. This replacement brings about a negative charge excess.

In the interior, the negative charges of the silicon-aluminum tetrahedra are compensated by positive ions of the alkali and alkaline-earth elements (Ca, K, Na, Mg) as well as by water.

A characteristic feature of the zeolites is selective activity in the replacement of these fixed ions by others (selectivity: Cs⁺>Pb²⁺>NH₄⁺>Cu²⁺>Hg²⁺>Cd²⁺>Ni²⁺>Co²⁺, NH₄⁺>K⁺>Mg²⁺>Ca²⁺). The ion-exchange capacity is 1.2-1.5 mol/kg. Na⁺ and K⁺ are the most dominant exchangeable cations of the zeolite.

They can be replaced by other elements and also, as is shown in the experiments in what follows, by harmful elements such as lead, ammonium, mercury and other heavy metals.

The surprising action of the clinoptilolite treated according to the invention consists in that, on the other hand, an essential element such as zinc is adsorbed only slightly or not at all.

In the quantities administered, the clinoptilolite is not toxic and, as further experiments show, is again completely excreted from the human organism along with the substances fixed by the process described above.

According to the invention, the clinoptilolite was subjected to a special form of treatment whereby it was reduced in size, activated and modeled.

The experiments on the effectiveness of the zeolite were preceded by investigations of some important particle properties and parameters such as the specific surface area, the particle-size distribution and the (potential of the activated clinoptilolite.

The experimental determination of the specific surface area was performed with an Areameter II instrument made by Juwe Laborgeräte GmbH, which makes it possible to determine specific surface area using a single-point method according to DIN 66132 as simplified by R. Haul and G. Dümbgen. The principle of measurement is based on low-temperature nitrogen adsorption. The specific surface area of the zeolite according to the invention was determined to be 35.961 m²/g.

The particle-size distribution was determined by laser diffraction using a Mastersizer 2000 instrument made by Malvern. The particle-size measurement was done by the laser diffraction method using a Mastersizer 2000 instrument made by Malvern. The clinoptilolite powder according to the invention was investigated in a particle-size range of 0.2-20 μm. The principal fraction of the particles was found to be in the particle-size range of 6-9 μm.

The ζ potentials were performed with the Zetamaster instrument made by Malvern GmbH. The ζ potentials were found to lie in the range of 24-26 mV.

The objective of one study performed was thus to simulate in vivo systems in vitro and to investigate quantitatively the above-described interaction of heavy-metal ions with the zeolite clinoptilolite. The model systems selected were chemical solutions similar in composition to gastric acid and the duodenal milieu, respectively. The metal ions under consideration were those of mercury (Hg²⁺), lead (Pb²⁺), cadmium (Cd²⁺), iron (Fe³⁺) and zinc (Zn²⁺) and additionally ammonium (NH₄⁺).

With regard to oral application, the concept of the invention consists in that, after intake, particles of clinoptilolite having the particle sizes attained through the treatment act in the human or animal digestive tract without penetrating into the cells. This means that the activated and modified clinoptilolite (MAC) is not resorbed in the human or animal gastrointestinal tract and can deploy its action chiefly there. The lack of resorption was demonstrated by experiments with technetium-99. The isotope scanner revealed no radiation in the organs that would have been caused by resorption of clinoptilolite particles tightly bound to the technetium-99.

In what follows, the in vitro detoxification experiments are described and the most important results are set forth.

The following mixture served as a synthetic gastric acid model solution:

| | |
|---|---|
| Pepsin | 1.16 g |
| KH₂PO₄ | 2.24 g |
| CaCl₂•2H₂O | 0.69 g |
| MgSO₄•7H₂O | 0.42 g |
| NaCl | 4.00 g |

This mixture was made up to 1 liter with HCl, the solution pH being adjusted to 1.5.

For the synthetic duodenal fluid, the above-described composition was used except for KH₂PO₄, because in the alkaline milieu this would lead to uncontrolled precipitation and coprecipitation of heavy-metal phosphates and hydroxides. The solution pH was adjusted to 8.1 with NaOH.

The heavy metals and ammonium under investigation were added to the synthetic gastric acid and the synthetic duodenal fluid from standard solutions to yield the following composition:

| | | |
|---|---|---|
| Fe | 10 | mg/L |
| Zn | 9 | mg/L |
| Pb | 0.9 | mg/L |
| Cd | 0.9 | mg/L |
| Hg | 0.9 | mg/L |
| NH₄ | 20 | mg/L |

Of each of the solutions so prepared, 100 mL portions were reacted with clinoptilolite without further conditioning in quantities of 0 g, 0.1 g, 0.2 g, 0.3 g, 0.4 g and 0.5 g. The suspensions were agitated for 90 minutes in a PTFE vessel. After the end of agitation the suspensions were centrifuged; the clear supernatant fluid served as the measurement solution for the individual determinations.

The methods used to determine the metal concentrations in the solutions were the ICP-OES method (Varian VISTA Pro) for Fe, Cd, Zn and Pb, and the cold vapor-AAS method (MWS DMA 80) for Hg.

Ammonium was determined by photometry at 655 nm (LP2W filter photometer).

In order to determine the acid-fixing capacity, 0.5 g of clinoptilolite was treated with 100 mL of synthetic gastric acid under the above-described conditions, the clinoptilolite was separated and the free acid was titrated with 0.1 N NaOH solution. A similar procedure was followed with an untreated gastric acid.

Experiments on the adsorption behavior of lead at pH 1.5 show that marked adsorption of lead takes place at pH 1.5. When the inlet quantity of the clinoptilolite is 0.5 g, 32% of the inlet lead is absorbed.

Very strong adsorption of lead similarly occurs in corresponding experiments in the synthetic duodenal solution at pH 8.1. When the inlet quantity of the clinoptilolite is 0.5 g, 86% of the inlet lead is adsorbed. In addition to true adsorption, coprecipitation of lead hydroxide with the iron hydroxide complexes may be responsible for very good separation.

Experiments on the adsorption behavior of mercury show good adsorption in the synthetic gastric acid at pH 1.5. When the inlet quantity of the clinoptilolite is 0.5 g, 57% of the inlet mercury is separated.

Experiments on the adsorption behavior of mercury in the synthetic duodenal model solution at pH 8.1 show that the adsorption of mercury is less good than at pH 1.5. When the inlet quantity of the clinoptilolite is 0.5 g, however, 45% of the mercury is still separated.

Along with the heavy metals, the adsorption behavior of the clinoptilolite according to the invention (MAC) in relation to ammonium ion was also determined. Experiments at pH 1.5 show no adsorption of ammonium ion dependent on the quantity of clinoptilolite applied.

In contrast, the experiments at pH 8.1 show marked adsorption of ammonium ion. When the inlet quantity of clinoptilolite is 0.5 g, 36% of the ammonium is separated. One cause may be that the ammonia/ammonium equilibrium (NH₃NH₄⁺) at pH 8.1 is shifted in favor of ammonia (NH₃), which is deposited on the clinoptilolite surface.

A further important part of the investigations was concerned with the question whether essential elements are not also adsorbed, which would lead to dangerous deficiency phenomena upon prolonged intake.

The adsorption behavior of iron in the synthetic gastric acid model solution at pH 1.5 was determined first. The experiments show that iron is given off from the clinoptilolite into the solution at this pH value. An increase in the iron concentration in dependence on the quantity of the clinoptilolite used can be clearly detected. When the inlet quantity of the clinoptilolite is 0.5 g, the increase in the iron concentration is 11% relative to the starting concentration.

Similarly to the foregoing experiments, the adsorption behavior of iron ions was also tested in the synthetic duodenal solution at pH 8.1. These experiments show that the iron concentration in the solution decreases at pH 8.1. True adsorption of Fe²⁺ or Fe³⁺, respectively, is improbable at pH 8.1; instead, an aggregation of iron hydroxy complexes from the clinoptilolite may be occurring. When the inlet quantity of the clinoptilolite (MAC) is 0.5 g, the decrease in iron in the solution is 14% relative to the starting concentration.

The behavior with respect to zinc as a further essential element was likewise investigated. Surprisingly, measurable adsorption in the synthetic gastric acid at pH 1.5 was seen only when larger quantities of the clinoptilolite were used. When the inlet quantity of the clinoptilolite was 0.5 g, roughly 1% of the zinc was absorbed.

For equal inlet quantities of the clinoptilolite, the adsorption of zinc is likewise low in the synthetic duodenal solution. Here the adsorption of the inlet zinc is only 1.6% when the inlet quantity of the clinoptilolite is 0.5 g.

In order to determine the acid-fixing capacity, a solution of synthetic gastric acid was agitated with 0.5 g of clinoptilolite, the clinoptilolite was separated and the free acid was then titrated with 0.1 N NaOH. An untreated gastric acid was analyzed for comparison:

| | |
|---|---|
| Untreated gastric acid | 57.1 mmol HCl |
| Gastric acid treated with 0.5 g clinoptilolite | 55.9 mmol HCl |

In order to verify this result, a pure hydrochloric acid was investigated in similar fashion. Here the untreated solution initially contained 93.7 mmol HCl. The HCl solution treated with 0.5 g of clinoptilolite contained only slightly less acid, 91.8 mmol. These results permit the inference that the clinoptilolite exhibits no significant acid-fixing capacity.

In summary, surprisingly, it can be stated that the investigations at pH 1.5 (simulated gastric milieu) show that the clinoptilolite has an extraordinarily good ability to adsorb lead (32%) and mercury (57%), while zinc (1%) and ammonium (1%) remain unaffected. For iron, a slight rise in the concentration in the synthetic gastric acid can be noted, the cause of which should be sought in the dissolution of iron from the clinoptilolite.

At pH 8.1 (simulated duodenal flora), lead (86%) and mercury (45%) are likewise well adsorbed, while zinc shows no reaction. The good adsorption of ammonium (36%) in this milieu is astonishing. Small portions of iron can likewise be absorbed.

It has thus been found that this specially treated clinoptilolite (MAC) according to the invention does indeed have enormous capacities for fixing heavy metals and ammonia and transporting them out of the human body. No adsorption capacity for the essential element zinc is to be observed.

The zeolite activated and modified according to the invention is successfully employed as a pharmaceutical agent and medicinal product against food intolerance. Applications outside the human digestive tract with a non-oral form of administration are also possible. Thus the zeolite, in a further embodiment, has also proved its worth as a skin powder for wound healing.

Similarly, burdens of heavy metals also arise through dental treatments, for example in the removal of amalgam fillings or the like.

In what follows, activated and modified zeolite is to be understood as denoting a zeolite whose particles exhibit a particle size of 2-9 μm, zeta potentials of 20-26 mV and a specific surface area of 30-38 m²/g.

An activated and modified zeolite is used as a pharmaceutical agent for diseases of the metabolic organs, in particular the liver, the kidneys and the pancreas, through the adsorption of heavy metals such as lead and mercury and/or ammonium in the digestive tract of human beings and animals and their expulsion from the organism.

A further use as a pharmaceutical agent is against food intolerance and food allergies in human being and animal. In this case the pharmaceutical agent is administered orally.

For the treatment of open or closed wounds, an activated and modified zeolite is used as a pharmaceutical agent in powder or cream form, the pharmaceutical agent being used externally and applied to the affected areas of the skin, the particles of the zeolite exhibiting a particle size of 6-9 μm, zeta potentials of 24-26 mV and a specific surface area of 35-36 m²/g.

A further use of an activated and modified zeolite as a pharmaceutical agent is in case of skin diseases and viral infections.

The inventive concept also comprises use of the activated and modified zeolite as a cosmetic agent for human and animal skin care and skin regeneration, the particles of the zeolite here again exhibiting a particle size of 2-9 μm, a zeta potential of 20-26 mV and a specific surface area of 30-38 m²/g.

A use of an active and modulated zeolite as a medicinal product for absorbing and expelling mercury and amalgam in the human tooth and jaw region consists in that when amalgam is removed, temporary fillings having the zeolite according to the invention are performed or the fillings exhibit constituents of the zeolite, these fillings being removed along with the absorbed and adsorbed undesired substances after a transient residence in the tooth or the jaw region. Here again, the particles of the zeolite exhibit a particle size of 2-9 μm, zeta potentials of 20-26 mV and a specific surface area of 30-38 m²/g.

1. Use of an activated and modified zeolite as a pharmaceutical agent for diseases of the metabolic organs, in particular the liver, the kidneys and the pancreas, through the adsorption of heavy metals such as lead and mercury and/or ammonium in the digestive tract of human beings and animals and their expulsion from the organism, the particles of the zeolite exhibiting a particle size of 2-9 μm, zeta potentials of 20-26 mV and a specific surface area of 30-38 m²/g.

2. Use of an activated and modified zeolite as a pharmaceutical agent against food intolerance and food allergies in human being and animal, the particles of the zeolite exhibiting a particle size of 2-9 μm, zeta potentials of 20-26 mV and a specific surface area of 30-38 m²/g.

3. Use of an activated and modified zeolite as a pharmaceutical agent in powder or cream form for the treatment of open or closed wounds, the particles of the zeolite exhibiting a particle size of 6-9 μm, zeta potentials of 24-26 mV and a specific surface area of 35-36 m²/g.

4. Use of an activated and modified zeolite as a pharmaceutical agent for skin diseases and viral infections, the particles of the zeolite exhibiting a particle size of 2-9 μm, zeta potentials of 20-26 mV and a specific surface area of 30-38 m²/g.

5. Use of an activated and modified zeolite as a cosmetic agent for skin care and skin regeneration of human being and animal, the particles of the zeolite exhibiting a particle size of 2-9 μm, zeta potential of 20-26 mV and a specific surface area of 30-38 m²/g.

6. Use of an active and modulated zeolite as a medicinal product for absorbing and extracting mercury and amalgam in the tooth and jaw region of the human being, the particles of the zeolite exhibiting a particle size of 2-9 μm, zeta potentials of 20-26 mV and a specific surface area of 30-38 m²/g.
